(19) United States
(12) Patent Application Publication
(10) Pub. No.: US 2025/0359971 A1
(43) Pub. Date: Nov. 27, 2025

(54) AN INTRAORAL SCANNER INCLUDING AN IMPROVED POWER MANAGEMENT UNIT

(71) Applicant: 3SHAPE A/S, Copenhagen K (DK)

(72) Inventors: Michael PEDERSEN, Copenhagen K (DK); Jonas Sejr BORCH, Copenhagen K (DK)

(73) Assignee: 3SHAPE A/S, Copenhagen K (DK)

(21) Appl. No.: 18/872,316

(22) PCT Filed: Jun. 7, 2023
(86) PCT No.: PCT/EP2023/065212

§ 371 Date: Dec. 6, 2024

(30) Foreign Application Priority Data
Jun. 7, 2022 (EP) 22177521

(51) Int. Cl. A61C 9/00

(52) U.S. Cl. CPC A61C 9/006; A61C 2204/002

(57) ABSTRACT

An intraoral scanner includes a projector unit configured to emit light at least onto a dental object of a patient; an image sensor configured to acquire reflected light from at least the dental object; a battery for powering the intraoral scanner; a processor unit configured to process the reflected light into one or more 2D images and/or 3D images; a wireless interface configured to communicate with an external device the one or more 2D images and/or 3D images, a motion sensor configured to sense a motion of the intraoral scanner; a timer unit; and a power management unit configured to reduce the power consumption of the intraoral scanner based on a motion signal provided by the motion sensor and a timer signal provided by the timer unit.

Buchanan

### FIELD

The disclosure relates to an intraoral scanner including an improved power management unit. In particular, the power management unit is configured to reduce the power consumption based on whether the intraoral scanner is being moved.

### BACKGROUND

A wireless handheld intraoral scanner is used for scanning a patient's mouth and capturing real-time images of the patient's mouth. The images are then transmitted real-time to an external device which is then used for generating surface information of the patients dentition and displaying the images or surface information in real-time. The images being transferred may be either raw 2D data captured by the scanner, processed 2D data or 3D data that includes multiple 2D data information. The transmission of the data is done via a wireless interface and the powering of the scanner is done via a battery that is connected to the scanner. The scanner needs significant amount of power to perform the scanning procedure of capturing multiple images, processing of the images and the transmission of the images to an external device. Furthermore, the whole process must be in real time contentiously for a long duration of time. The dentist is typically operating the scanner with one hand and a scanning session may take between several minutes and an hour depending on the treatment. The ergonomic usage of the scanner puts restriction on the total weight of the scanner device, and therefore, it would not be ideal to just increase the size of the battery to obtain the needed operation time of the scanner before a new battery is needed or charging of the battery is needed. Furthermore, the intraoral scanner should be easy to handle by one hand and the size of the scanner is also restricted to the size of the mouth of the patient, and again, it is not possible to just increase the size of the battery. Typically, a dentist uses the scanner during multiple consecutive patient visits requiring the scanning device to be constant ready to use without the need to constantly switch battery when depleted. Therefore, there is a need for having a power management unit that can improve the power consumption of the scanner to fulfill the need of having a lightweight and small sized scanner without compromising the battery life.

Additionally, the scanner includes more functionalities than described, and all of these including the previous mentioned functionalities are activated during a scanning. During a typical scanning workflow user statistic have shown that at a dental clinic the actual scanning time represents a small fraction of the total treatment time. Long periods of preparing the patient for scanning extents the total scanning sessions. This leads to unnecessary battery consumption keeping the scanner ready during the entire session. In order to extent the battery life as much as possible, a number of low power mode are needed for the scanner to save power between actual scanning performance while always making the scanner capable of rapidly restart scanning.

### SUMMARY

An aspect of the present disclosure is to reduce the power consumption of the intraoral scanner by for example reducing leakage current through the electronic components of the scanner.

According to the aspects, an intraoral scanner is disclosed. The intraoral scanner may include a projector unit configured to emit light at least onto a dental object of a patient and an image sensor configured to acquire reflected light from at least the dental object.

The intraoral scanner may include a light passing region in the distal end where the light passes through to and from the object to be scanned. The light passing region may include a optically transparent element with or without a λ/4 or a λ/2 waveplate for polarizing the light and/or for polarizing the reflected light that passes through the window and onto the image sensor. The intraoral scanner may include a battery for powering the intraoral scanner. The battery may be configured to be detachable mounted to the scanner via a battery interface of the scanner to enable fast replacement of battery if one battery is depleted. The battery may be charged when being mounted to the scanner or when removed from the scanner and placed into a dedicated charger device. The battery may include a processor unit and firmware stored on a memory of the battery. A communication between a power management unit of the scanner and the battery may be established for communicating charge capacity, battery errors or other type of battery information. The intraoral scanner may include a processor unit configured to process the captured 2D images of the reflected light into one or more 2D images and/or 3D images. In another example, the processor unit may be configured to forward the raw data received and/or processed to an external device via a wireless interface of the scanner. The wireless interface may be configured to communicate with an external device to transmit the one or more 2D images and/or 3D images. The scanner may include a motion sensor configured to sense a motion of the intraoral scanner. The motion sensor may include an accelerometer and/or a gyroscope. The intraoral scanner may include a timer unit. The timer unit may be part of the processor or any components of the scanner. The timer unit may be a processor clock that is configured to determine how quick the processor unit can retrieve and interpret instructions. The intraoral scanner may include a power management unit configured to reduce the power consumption of the intraoral scanner based on a motion signal and a timer signal. Wherein the motion signal includes information about motion of the intraoral scanner, wherein the motion may be below a motion threshold during the one or more periods, and after each of the one or more time periods the power consumption is reduced gradually. During a scanning session all components of the scanner is turned on and during the use of the scanner, the motion unit detects movement in many different directions as the dentist typically moved the scanner around in the intraoral cavity to obtain surface information from multiple angles and positions to cover the dentition. The user decides to pause the scanning session by placing the scanner onto a table or a cradle, i.e. into a passive position, where no movement or approximately no movement is detected by the motion unit. In this situation, the power management unit is then configured to reduce the power consumption by for example stepwise turning off more and more components as the one or more time periods elapses by selectively stepwise disabling the power supply from the battery to the components.

The power consumption reduction may include setting the intraoral scanner into different power modes based on the motion signal and the timer signal after each of the one or more time periods. For example, when a first time period has elapsed, the power management unit is configured to set the intraoral scanner into a first power mode resulting in a first lowering of the power consumption of the scanner. Following the first time period, a second time period elapses and the power management unit is configured to set the scanner into a second power mode which results in a further lowering of the power consumption of the scanner.

The power management unit may be configured to set the scanner into a plurality of power modes.

The power consumption reduction may include gradually turning off components of the intraoral scanner after each of the one or more time periods. For example, the power management unit may be configured to turn off several components of the intraoral scanner after a first time-period with no or about no movement of the scanner by disabling the supply of power from the battery. The power management unit may be configured to turn off additional components of the intraoral scanner after a second time-period consecutive to the first time-period. By completely turning off components and not placing them into a low power state results in a heavily reduction of leakage current. The leakage current is not avoided by just placing a component into a low power state as some current will leak through the individual electronic components if they are supplied power. Thereby, the power consumption is even more reduced in comparison to when just placing the components into a low power configuration which may be pre-programmed by the component manufacturer.

The power consumption reduction may include gradually turning off one or more groups of components of the intraoral scanner after each of the one or more time periods, and wherein each of the one or more groups includes one or more components of the intraoral scanner. After a time period a group of components is turned off and/or put into a low power mode. Some of the components of the group are turned off and some are set into a low power mode. For example, the scanner may include a heater that is configured to heat the window of the scanner in order to avoid fog being created on the window during a scanning session in the humid environment of the intraoral cavity. It would not be ideal to completely turn off the heater as the scanner would not be immediate ready for use if the user wants to restart the scanner procedure after one or more time-period of no or about no movement of the scanner.

A component of the intraoral scanner may be a projector unit, an image sensor, a processor, a wireless interface, a motion sensor, a part of the motion sensor, timer unit, part of the power management unit, a temperature sensor, a heater, a user interface, a part of the user interface, a vibrator, a fan for cooling components or for transferring heat from components to the window, LED, UV LED, white LED, IR LED, NIR LED, a motor for changing positions of optical components of the scanner, memory, flash and other components that are used for driving, controlling and powering the scanner.

The intraoral scanner may include components and/or one or more groups of components, and the components include at least the projector unit, the image sensor, processor unit, the wireless interface, the motion sensor, the timer unit, and the power management unit.

The motion threshold defines a threshold of movement that may be characterized as no movement or about no movement. In practice, there will always be some level of movements even though the scanner is not being used.

The different modes may include one or more of following:
a scanning mode, wherein the motion signal includes information about motion of the intraoral scanner being above the motion threshold,
an idle mode, wherein the motion signal includes information about motion of the intraoral scanner that is below the motion threshold during a first time-period,
a sleep mode, wherein the motion signal includes information about motion of the intraoral scanner that is below the motion threshold during a second time period, wherein the second time period is longer than the first time period, and
a deep sleep mode, wherein the motion signal includes information about motion of the intraoral scanner that is below the motion threshold during a third time period, wherein the third time period is longer than the second time period, and
wherein the first time-period, the second time-period and the third time-period are part of the one or more time periods.

For example, in the deep sleep mode more components are turned off, not supplied with power, and/or set into a low power mode in relation to the sleep mode and the idle mode. More components are turned off and/or set into a low power mode in relation to the idle mode. It's an advantage to turn off the components rather than set them to a low power mode if wanting to achieve best possible power consumption during no use of the scanner. However, it is of an advantage to combine turning off components and setting one or more components into a low power mode if a faster transition from idle, sleep, deep sleep to the scanning mode is wanted. In the scanning mode, the scanner is ready to be used.

In the scanning mode all components are turned on.

In deep sleep mode, only the gyroscope and the pushbutton (i.e. the user interface) are on and will trigger the power management unit to power up the scanner. From this mode some boot time is expected to power up and establish the wireless connection.

In off mode every component of the scanner is unpowered. Scanner is woken by activation of scanner user interface, e.g. a pushbutton. This will bring the scanner to the sleep state.

In a no power mode, the scanner battery or smart cord is disconnected, and there is no power on the scanner. In the idle mode at least the projector unit, the image sensor and the processor unit are turned off.

Additionally, in the idle mode a heater for defogging the distal end of the intraoral scanner may be configured to heat at a lower temperature than in the scanning mode. By enabling a lowering the heating temperature results in a faster startup of the scanner when being in the scanning mode or in a faster transition to the scanning mode where the scanner is ready to be used.

The sleep mode may include that at least the image sensor, the projector unit, the processor unit and the heater are turned off. The power consumption is even more reduced in view of the idle mode.

The deep sleep mode may include that at least the projector unit, the image sensor, the processor unit, the wireless interface, and part of the motion sensor are turned off. In this example, the gyroscope is turned off and the accelerometer is on. Thereby, the power consumption is even more reduced, but the motion sensor is still able to sense movement of the scanner.

The intraoral scanner may include a user interface that is configured to provide a user interface signal to the power management unit, and the power management unit is configured to set the intraoral scanner into the scanning mode when receiving the user interface signal. This may be possible only if the scanner is in sleep mode or in deep sleep mode.

The user interface signal may be provided by a button press, a touch pad press, a capacitive coupling between a user's finger, an acoustical signal etc.

When the motion is above the motion threshold, the power management unit is configured to set the intraoral scanner into the scanning mode if the intraoral scanner is in the idle mode.

The power management unit is configured to set the scanner into the scanning mode when receiving a first signal and/or a second signal.

The user interface may be configured to provide a user interface signal to the power management unit, and the power management unit may be configured to set the intraoral scanner into the scanning mode if the motion is above the motion threshold during a motion time period and if receiving the user interface signal. Thereby, if the user is walking with the scanner in its hand, then the scanner will not be set into the scanning mode until the user interacts with the user interface.

The power management unit may be configured to set the scanner into the scanning mode if the motion detected by the motion sensor corresponds to a predefined direction or pattern that relates to an intentionally use of the intraoral scanner. The pattern and/or the predefined direction may be stored in a memory of the scanner.

The power management unit supports different power domains, pushbutton wake up, sequencer, ADC's, GPIO's for various purposes, that will integrate well into the intraoral scanner. The user interface, e.g. a push-button controller, and a main load switch of the scanner are powered directly from the battery connector. The rest of the system is powered through the main load switch, including the power management unit which controls most of the power system and the power modes.

In the off mode, the main load switch is turned off, and only the user interface is powered (or no battery is present). In the deep sleep mode, the main load switch is turned on, and the power management unit is in a state, where only the gyroscope is powered by the power management unit.

When a battery is inserted into the scanner, the push-button controller will turn on the main load switch and thereby power the power management unit. The power management unit will start it's power sequence and power the system up to the sleep mode, where a control unit of the scanner is powered up and booted. From here the control unit is controlling which power state to go to. If a scanning is requested, the control unit will command the power management unit to go to the scanning mode.

If the scanner has been unused for a while in the sleep mode, the control unit can decide to command the power management unit to go to the deep sleep mode. The control unit is thereby powered off, and the power management unit is only powering the gyroscope. From the deep sleep state the scanner can power up to the sleep state in two different ways. The first way is when the gyroscope detects, that the scanner is being moved. The gyroscope will enable a wake-up signal directly to the power management unit, which will then power the system up to the sleep state. The second way is when the user interacts with the user interface, such as by pushing a primary button. The push-button controller will detect the button push, and enable a wake-up signal directly to the power management unit, which will then power the system up to the sleep mode.

From the sleep mode, the control unit may command the push-button controller to turn off the main load switch. That will power off the whole system except the push-button controller, and the scanner may then be in the power off mode. From there only a button push on the primary button can wake up the scanner. The push-button controller will turn on the main load switch and thereby power the power management unit. The power management unit will start it's power sequence and power the system up to the sleep state. In some situations it would not be appreciated that the scanner enters a deep sleep mode or an off mode based on the motion and timer, and therefore, the power management unit is configured to set the intraoral scanner into a master power mode which prevents the intraoral scanner to enter a low power mode, such as the idle mode, sleep mode, the deep sleep mode and/or an off mode. The master mode may be set via a user interface of the scanner or via an external device, such as a computer, that communicates wirelessly or wired to the intraoral scanner. In the master mode, the user would never experience waiting time for the scanner to be in the scanner mode after it has been in a low power mode.

The time period(s) may be customized by a user of the intraoral canner via an external and the wireless interface. For some users it may be appreciated if the scanner enters a low power mode earlier or later than for other users, thereby, the user would not experience frustrations in the daily use of the scanner when entering a low power mode. It's a main benefit of the invention to create a hibernation mode without creating any frustrations, and the dentist should experience the scanner as always ready.

The intraoral scanner may include a temperature sensor for measuring the heat inside the scanner. The scanner must not be too warm for the purpose of not damaging components within the scanner as well as keeping the outside shell temperature below specific temperature limits required for medical devices. The scanner may include a haptic transducer unit configured to apply a tactile feedback such as a vibration to the intraoral scanner during the one or more time periods. The haptic transducer may be activated when the motion sensor detects that the intraoral scanner is moving. The vibration may be activated shortly and with a time interval. The activation may be between 1 and 10 seconds, and the time interval between each activations may be between 20 seconds and 180 seconds. This is of particular advantage when the scanner is “waking up” from a low power state to enter a higher power mode. When the scanner is in one of the lowest power modes, many functions of the scanner might be switched off including the fan or LED ring or any other components that may provide some form of feedback to the dentist about that the scanner is alive and working. In this case a short vibration will notify the user that the scanner is alive and about to enter higher power state with multiple functions ready.

The power management unit is configured to set the intraoral scanner into a battery level mode when a charge level of the battery is below a charge level threshold. That will prolong the operation time of the scanner with the battery being low on power. The battery level mode includes one or more of following settings of the intraoral scanner in comparison to the scanning mode:
a lowering of a data rate of the wireless interface;
a lowering of the lumens of the projector unit;
a lowering of a heating temperature provided by a heater of the intraoral scanner, and
a lowering of processing speed provided by the processor unit.

The intraoral scanner may comprise a user interface including one or more LEDS, wherein the color of the one or more LEDS or a frequency of flashing of the one or more LEDS corresponds to the charge level of the battery.

The intraoral scanner may comprise a user interface that includes at least a button configured to forward a short button press signal or a long button press signal to the power management unit, and the short button press signal corresponds to a button press that occurs during a time period that is shorter than for the long button press signal, and wherein the short button press signal corresponds to a turn on of the intraoral scanner and/or to set the intraoral scanner into a scanning mode, and the long button press signal corresponds to a turn off of the intraoral scanner.

The power management unit may be configured to receive a master signal that determines the power mode of the intraoral scanner irrespective of the motion signal and the timer signal.

The master signal may include one or more of following signals:
a wireless signal received by the wireless interface, and wherein the wireless signal includes information about lost connection to an external device;
a wireless interface signal provided by the wireless interface, and wherein the wireless interface signal includes information about lost wireless connection; and
a temperature signal provided by a temperature sensor of the intraoral scanner, and wherein the temperature signal includes information about the temperature of the intraoral scanner that is higher than a temperature threshold.

According to the aspects, an intraoral scanner is disclosed. The handheld intraoral scanner comprising:
a projector unit configured to emit light at least onto a dental object of a patient;
an image sensor configured to acquire reflected light from at least the dental object;
a battery for powering the scanning device
a wireless interface configured to communicate with an external device,
a motion sensor for sensing a motion of the intraoral scanner; and
a power management unit configured to set the intraoral scanner into a plurality of power modes based on a motion signal provided by the motion sensor; and
wherein the plurality of power modes includes:
   an activation mode when the motion signal includes information about a movement of the intraoral scanner during a first time period,
   a power down mode when the motion signal includes information about no movement of the intraoral scanner during a second time period,
   a sleep mode when the motion signal includes information about no movement of the intraoral scanner during a third time period, wherein the third time period is longer than the second time period, and
   a deep sleep mode when the motion signal includes information about no movement of the intraoral scanner during a fourth time period, wherein the fourth time period is longer than the third time period.

According to the aspects, an intraoral scanner is disclosed. The handheld intraoral scanner comprising:
a projector unit configured to emit light at least onto a dental object of a patient;
an image sensor configured to acquire reflected light from at least the dental object;
a wireless interface configured to communicate with an external device,
a motion sensor for sensing a motion of the intraoral scanner;
a timer unit; and
a power management unit configured to set the intraoral scanner into different power modes based on a motion signal provided by the motion sensor and a timer signal provided by the timer unit.

According to the aspects, an intraoral scanner is disclosed. The handheld intraoral scanner comprising:
a projector unit configured to emit light at least onto a dental object of a patient;
an image sensor configured to acquire reflected light from at least the dental object;
a processor unit configured to process the reflected light into one or more 2D images and/or 3D images;
a wireless interface configured to communicate with an external device the one or more 2D images and/or 3D images,
a motion sensor configured to sense a motion of the intraoral scanner;
a timer unit; and
a power management unit configured to reduce the power consumption of the intraoral scanner based on a type of information signal.

The type of information signal may be one or more of following:
a motion signal and a timer signal, and wherein the motion signal includes information about motion of the intraoral scanner during a time-period that is below a motion threshold, and while the time-period increases the power consumption is reducing;
a wireless signal received by the wireless interface, and wherein the wireless signal includes information about lost connection to an external device;
a wireless interface signal provided by the wireless interface, and wherein the wireless interface signal includes information about lost wireless connection; and
a temperature signal provided by a temperature sensor of the intraoral scanner.

According to the aspects, an intraoral scanner is disclosed. The handheld intraoral scanner comprising:
a projector unit configured to emit light at least onto a dental object of a patient;
an image sensor configured to acquire reflected light from at least the dental object;
a processor unit configured to process the reflected light into one or more 2D images and/or 3D images;
a wireless interface configured to communicate with an external device the one or more 2D images and/or 3D images,
a motion sensor configured to sense a motion of the intraoral scanner;
a timer unit; and
a power management unit configured to gradually turn off components of the intraoral scanner based on a motion signal and a timer signal.

The motion signal includes information about motion of the intraoral scanner during one or more time-periods that is below a motion threshold, and after each of the one or more time-periods the components are being turned off gradually.

According to the aspects, an intraoral scanner is disclosed. The handheld intraoral scanner comprising:
a projector unit configured to emit light at least onto a dental object of a patient;
an image sensor configured to acquire reflected light from at least the dental object;
a processor unit configured to process the reflected light into one or more 2D images and/or 3D images;
a wireless interface configured to communicate with an external device the one or more 2D images and/or 3D images,
a motion sensor configured to sense a motion of the intraoral scanner;
a timer unit; and
a power management unit configured to reduce the power consumption of the intraoral scanner based on a motion signal and a timer signal, and wherein the motion signal includes information about no motion of the intraoral scanner during a time-period, and efter the time-period the power consumption is reduced.

The motion signal includes information about movement of the intraoral scanner, the power management unit is configured to set the intraoral scanner into a scanning mode.

The power management unit is configured to set the intraoral scanner into a scanning mode when receiving an enablement signal from a user interface of the intraoral scanner.

The motion threshold corresponds to no movement.

The power management unit is configured to receive a master signal that determines the power consumption of the intraoral scanner irrespective of the motion signal.

An intraoral scanner may comprise a first sensor for sensing a first parameter indicative of a use situation of the intraoral scanner, the intraoral scanner defining a direction parallel to a direction when in a use situation, wherein the scanning device is configured to change into a high power mode or a low power mode in response to a first control signal from the first sensor, wherein the first sensor is a three-axis accelerometer having one axis aligned with the direction, and wherein the intraoral scanner changes from the high power mode to the low power mode by: measuring accelerations of the intraoral scanner in three directions from the first sensor; determining whether an orientation of the intraoral scanner deviates more than a threshold value from vertical; determining whether a total acceleration of the intraoral scanner deviates less than a predetermined value from earth's gravity; and changing the intraoral scanner from the high power mode to the low power mode when the orientation deviates more than the threshold value from vertical and the total acceleration deviates less than the predetermined value from earth's gravity in response to the first control signal from the first sensor.

The intraoral scanner may comprise a second sensor for sensing a second parameter indicative of the use of the intraoral scanner, and wherein the intraoral scanner is adapted to change into the high power mode or into the low power mode in response to a second control signal from the second sensor and/or the first control signal from the first sensor.

At least one of the first parameter and second parameter comprises one or more of: an orientation of the intraoral scanner relative to a predetermined axis, a movement of the intraoral scanner, an acceleration of the intraoral scanner, a humidity level and a temperature.

The intraoral scanner may be adapted to change between the high power mode and the low power mode in response to the first control signal and/or a second control signal.

The intraoral scanner may be adapted to change between the high power mode and the low power mode in response to a predetermined combination of the first and the second control signals.

One of the first and the second parameters may be an orientation of the intraoral scanner, and wherein the intraoral scanner may be adapted to change into the high power mode in response to a change in the orientation of the intraoral scanner into a none horizontal or a none partially horizontal orientation.

One of the first and the second parameters is an orientation of the intraoral scanner, and wherein the intraoral scanner is adapted to change into the low power mode in response to a change in the orientation of the intraoral scanner into a partially horizontal orientation.

The first sensor may be a tilt sensor that is adapted to determine an orientation of the intraoral scanner relative to a predetermined axis.

The first sensor may be a movement sensor that is adapted to determine movement of the intraoral scanner in at least one direction.

The intraoral scanner may comprise a humidity sensor that is adapted to determine the humidity in the vicinity of the intraoral scanner.

The time periods may be determined from the moment no movement is detected.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIGS. 1A and 1B illustrate an example of an intraoral scanner;

FIGS. 2A and 2B illustrate the reduction in power consumption;

FIGS. 3A and 3B illustrate an example of the intraoral scanner;

FIG. 4 illustrates an example of the intraoral scanner;

FIG. 5 illustrates an example of a power management unit;

FIG. 6 illustrates an example of the power management unit;

FIG. 7A, 7B, and 7C illustrate an example of the power management unit;

FIG. 8 illustrates an example of a state diagram of the power management unit; and

FIG. 9 discloses a table overview of different power modes.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, central processing units (CPUs) based on the reduced instruction set computer (RISC) architecture such as an ARM processor, System on a Chip (SoP) configurations of multiple processors such as a Zync processor, a graphics processing unit (GPU), neural processing units (NPU), Video Encoders, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intra-oral scan data may be performed by a dental scanning system that may include an intraoral scanning device such as the TRIOS series scanners from 3Shape A/S. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is capable of obtaining surface information by operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled relative to the dentition during a scanning session, such that at least some sets of sub-scans overlap at least partially, in order to enable reconstruction of the digital dental 3D model by stitching overlapping subscans together in real-time and display the progress of the virtual 3D model on a display as a feedback to the user. The result of stitching is the digital 3D representation of a surface larger than that which can be captured by a single sub-scan, i.e. which is larger than the field of view of the 3D scanning device. Stitching, also known as registration and fusion, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit. Color texture of the dental object may be acquired by illuminating the object using different monochromatic colors such as individual red, green and blue colors or my illuminating the object using multichromatic light such as white light. A 2D image may be acquired during a flash of white light.

Generally the process of obtaining surface information in real time of a dental object to be scanned requires the scanning device to illuminate the surface and acquire high number of 2D images. Typically a high speed camera is used with a framerate of 300-2000 2D frames pr second dependent on the technology and 2D image resolution. The high amount of image data needed to be handled by the scanning device to eighter directly forward the raw image data stream to an external processing device or performing some image processing before transmitting the data to an external device or display. This process requires that multiple electronic components inside the scanner is operating with a high workload thus requiring a high demand of current.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. Second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit or a wired network unit. The wireless network unit is configured to wirelessly connect the dental scanning system to the network comprising the plurality of network elements including the at least one network element configured to receive the processed data. The wired network unit is configured to establish a wired connection between the dental scanning system and the network comprising the plurality of network elements including the at least one network element configured to receive the processed data.

The dental scanning system preferably further comprises a processor configured to generate scan data (such as extra-oral scan data and/or intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

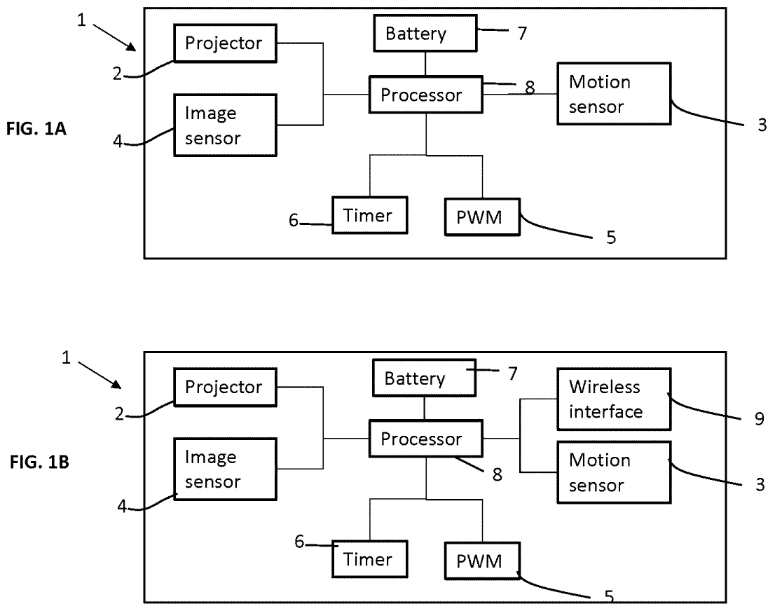

FIGS. 1A and 1B illustrate an example of an intraoral scanner 1. The intraoral scanner 1 comprises a projector unit 2 configured to emit light at least onto a dental object of a patient, an image sensor 4 configured to acquire reflected light from at least the dental object, a battery 7 for powering the intraoral scanner 1. Furthermore, the scanner 1 includes a processor unit 7 configured to process the reflected light into one or more 2D images and/or 3D images, a motion sensor 3 configured to sense a motion of the intraoral scanner 1 and a timer unit 6. Additionally, the intraoral scanner 1 includes a power management unit 5 configured to reduce the power consumption of the intraoral scanner 1 based on a motion signal provided by the motion sensor 3 and a timer signal provided by the timer unit 6. The motion signal includes information about motion of the intraoral scanner 1, wherein the motion is below a motion threshold during one or more periods, and after each of the one or more time periods the power consumption is reduced gradually. The intraoral scanner 1 may be configured to communicate wirelessly or wired to an external device (not shown). The communication may include images, status, firmware update charging power etc. In. FIG. 18, the intraoral scanner 1 includes a wireless interface configured to communicate with an external device the one or more 2D images and/or 3D images.

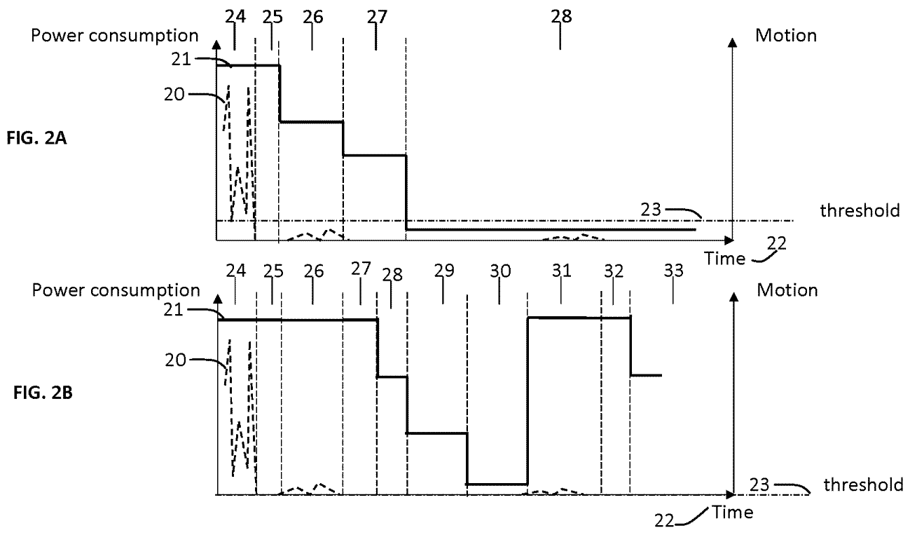

FIGS. 2A and 2B illustrates the reduction in power consumption of the intraoral scanner 1. In FIG. 2A, the timer signal 22 is depicted on the x axis of the, the motion signal 20 on a second y axis, and the power consumption 21 on a first y axis. In the example, motion is detected within a time period 24, and then during at least a first time period 25, the motion 20 has stopped or reduced to below a motion threshold 23, and at the end of the first time period 25, the power management 5 sets the intraoral scanner 1 into an idle mode. In the idle mode, the power management unit 5 is configured to at least turn off the projector unit 2, the image sensor 4, and the processor unit 8. The user may have customized the idle mode to include also a lowering of a heater within the scanner 1 to lower the temperature. Then, when being in the idle mode and after a second time period 26 with no motion or motion just below the threshold 23, the power management 5 sets the scanner 1 into a sleep mode that causes a further reduction of the power consumption of the scanner 1. In this mode, at least the image sensor 2, the projector unit 4, the processor unit 8 and the heater are turned off. Then, after a third time period 27 the power management unit 5 is configured to set the scanner 1 into a deep sleep mode wherein at least the projector unit 2, the image sensor 4, the processor unit 8, the wireless interface 9, and part of the motion sensor 3 are turned off. The motion sensor 3 includes a gyroscope and an accelerometer, and in the deep sleep mode, it is the gyroscope which is turned off. In this example, the scanner 1 is in the sleep mode during a fourth time period 28, but in another example, the power management unit 5 may be configured to set the scanner 1 into an off mode wherein all components, with the exception of a user interface of the scanner 1, would be turned off. The user interface is needed for turning on the scanner 1. The off mode may be set during a fifth time period subsequently to the fourth time period 28. During the fourth time period 28 a motion is detected by the motion sensor 3, but the motion is not above threshold 23, and therefore, the power management unit 3 does not set the scanner 1 into the scanner mode. In the different modes it is seen that the power consumption of the scanner 1 reduces.

In FIG. 2B, the motion threshold 23 is set to about 0, i.e., corresponding to about no motion. Similar to FIG. 2A, during the time period 24 motion is detected by the motion sensor, and during a first time period 25 no motion is detected. Then just before a first time period 25 elapses and during a second time period 26 a small motion is detected by the motion sensor 3, and the power management unit 5 does not reduce the power consumption. Then during a third, fourth and fifth time periods (27,28,29) no motion is detected and the power consumption is reduced gradually, and then just before the fifth time period 29 elapses and during a sixth time period 30 a small motion is detected and that is enough for the power management unit 5 to set the scanner 1 into a scanner mode.

The time periods may be determined from the moment no movement is detected.

When the scanner 1 enters different low power modes, e.g. the idle, sleep or deep sleep mode, the power management unit 5 is configured to turn off components of the intraoral scanner after each of the one or more time periods has elapsed. The settings of each modes, i.e. which includes turning off components or set a component to a low power mode, implies a trade-off between fast transition time going from a low power mode to the scanner mode and maximum reduction of power consumption of the scanner 1. The suggested settings of the low power modes have included the trade-off.

Each component of the intraoral scanner 1 may be turned off sequentially or in groups. The reduction of power consumption includes gradually turning off one or more groups of components of the intraoral scanner after each of the one or more time periods (25, 26, 27, 28, 29, 30, 31, 32, 33), and wherein each of the one or more groups includes one or more components of the intraoral scanner 1.

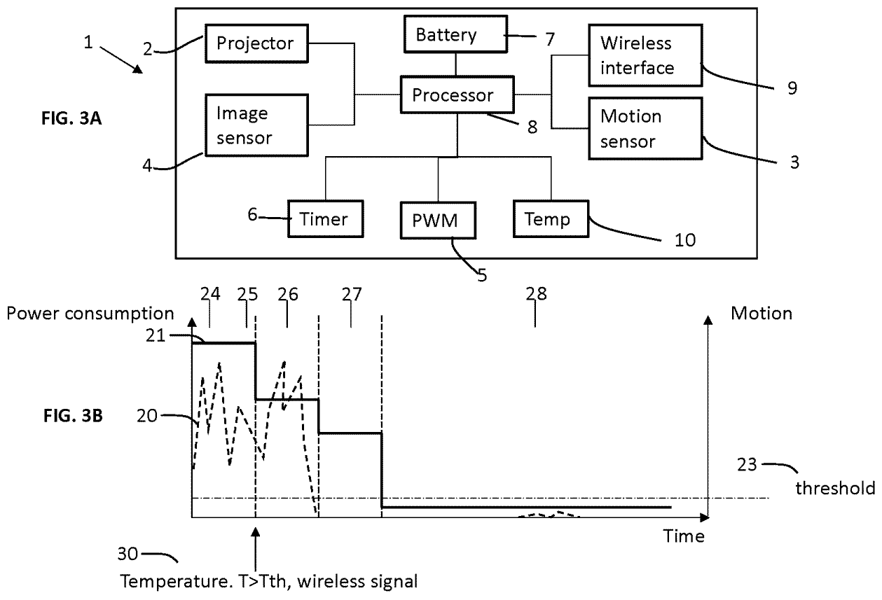

FIGS. 3A and 3B illustrate an example of the scanner 1 that includes a temperature sensor 10. In FIG. 3B, it is seen that the scanner entera a low power mode in the second time period 26 because of a detected temperature 30 being above a temperature threshold (Tth). In another example, the power management unit 5 is configured to set the intraoral scanner 1 into a low power mode while a motion is detected since a wireless signal is lost, meaning no WIFI or Bluetooth connection is available, or, the scanner 1 has lost connection to an external device.

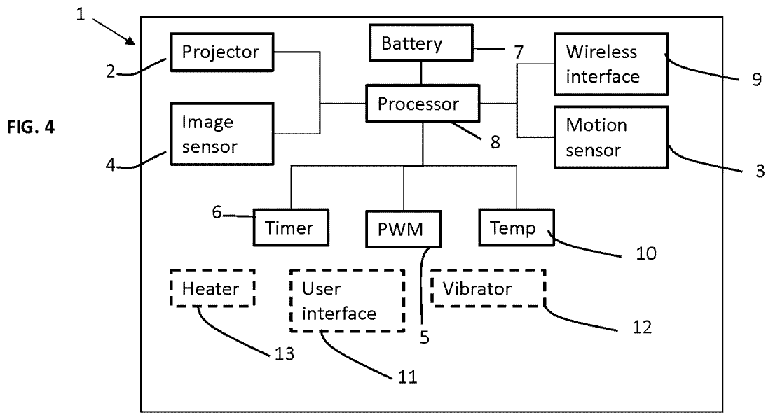

FIG. 4 illustrates the scanner 1 including a heater 13, a user interface 11 and a vibrator unit 12. The heater is configured to heat a scanning window of the scanner 1 arranged at a tip of the scanner 1 where the light from the projector unit 2 is projecting away from the scanner 1 and onto a dental object, such as tooth or gingival. The heating of the scanning window prevents fog from being created on the scanning window during scanning of a patient. In one low power mode, the heater may be either turned off or reduced to provide a lower heating temperature. The vibrator unit 12 is configured to apply a vibration to the intraoral scanner 1 during the one or more time periods. The vibrator unit 12 may be activated when the motion sensor 3 detects that the intraoral scanner 1 is moving. The vibrator 12 may be activated shortly and with a time interval. The activation may be between 1 and 10 seconds, and the time interval between each activations may be between 20 seconds and 180 seconds. The user interface 11 may include one or more buttons, touch pads, capacitive switch, or, the user interface 11 may be provided externally via an external device. For example, the user may control the scanner 1 via a computer or a smartphone that communicates wireless to the scanner 1. The user interface that includes at least a button is configured to forward a short button press signal or a long button press signal to the power management unit 5, and the short button press signal corresponds to a button press that occurs during a time period that is shorter than for the long button press signal. The short button press signal corresponds to a turn on of the intraoral scanner and/or to set the intraoral scanner into a scanning mode, and the long button press signal turns off the intraoral scanner.

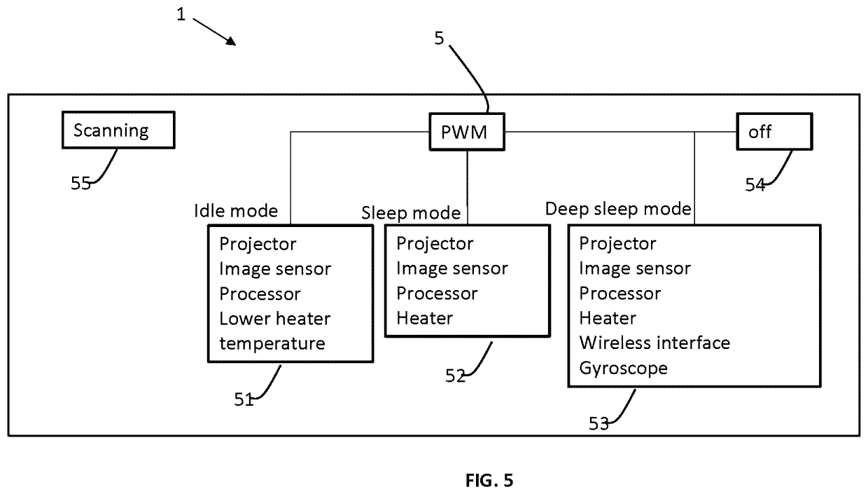

FIG. 5 illustrates the power management unit 5. In this example, the power management unit 5 is configured to set the intraoral scanner 1 into five different modes:
an off mode 54, wherein all components of the scanner are turned off except of the user interface 11,
a scanning mode 55, wherein the motion signal includes information about motion of the intraoral scanner 1 being above the motion threshold,
an idle mode 51, wherein the motion signal includes information about motion of the intraoral scanner 1 that is below the motion threshold during a first time-period,
a sleep mode 52, wherein the motion signal includes information about motion of the intraoral scanner 1 that is below the motion threshold during a second time period, wherein the second time period is longer than the first time period, and
a deep sleep mode 53, wherein the motion signal includes information about motion of the intraoral scanner that is below the motion threshold during a third time period, wherein the third time period is longer than the second time period, and
wherein the first time-period, the second time period and the third time period are part of the one or more time periods.

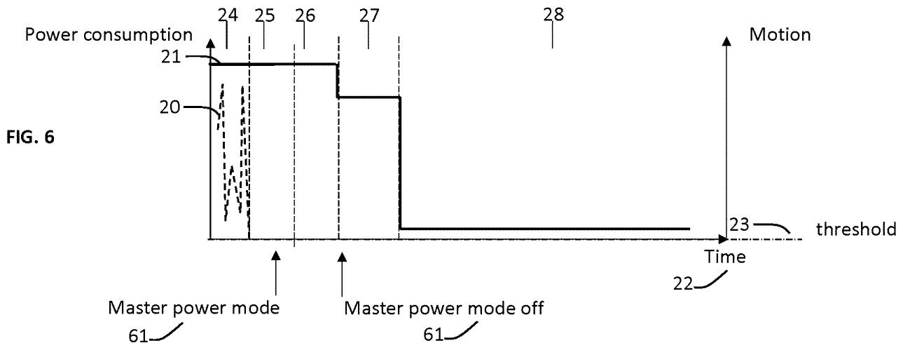

FIG. 6 illustrates an example of the power management unit 1 configured to set the intraoral scanner 1 into a master power mode 61 which prevents the intraoral scanner 1 to enter a low power mode, such as the idle mode, sleep mode, the deep sleep mode and/or an off mode.

In the example, no motion is detected from the first time period 25 but the scanner does not enter a low power mode as the master power mode 61 is turned on. The moment where the master power mode 61 is turned off, the scanner enters a low power mode if no movement is detected or the motion is below the motion threshold 23.

The power management unit 5 is configured to receive a master signal that determines the power mode of the intraoral scanner irrespective of the motion signal and the timer signal. The master signal includes one or more of following signals:
a wireless signal received by the wireless interface 9, and wherein the wireless signal includes information about lost connection to an external device;
a wireless interface signal provided by the wireless interface, and wherein the wireless interface signal includes information about lost wireless connection; and
a temperature signal provided by a temperature sensor 10 of the intraoral scanner, and wherein the temperature signal includes information about the temperature of the intraoral scanner 1 that is higher than a temperature threshold.

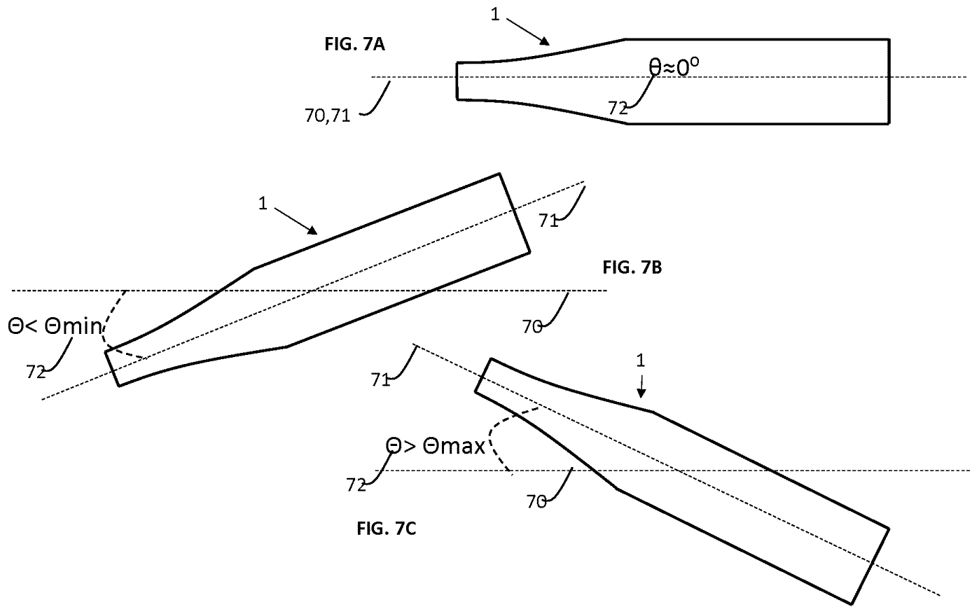

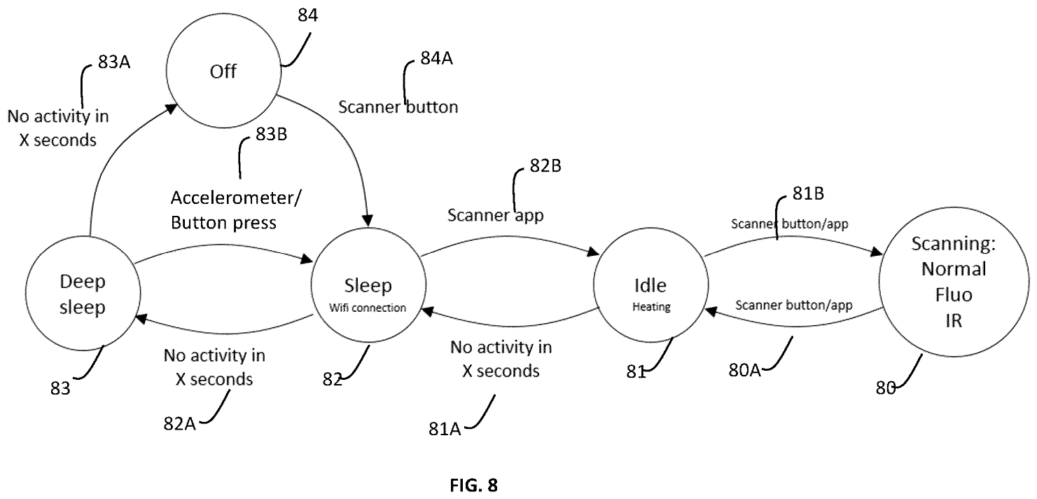

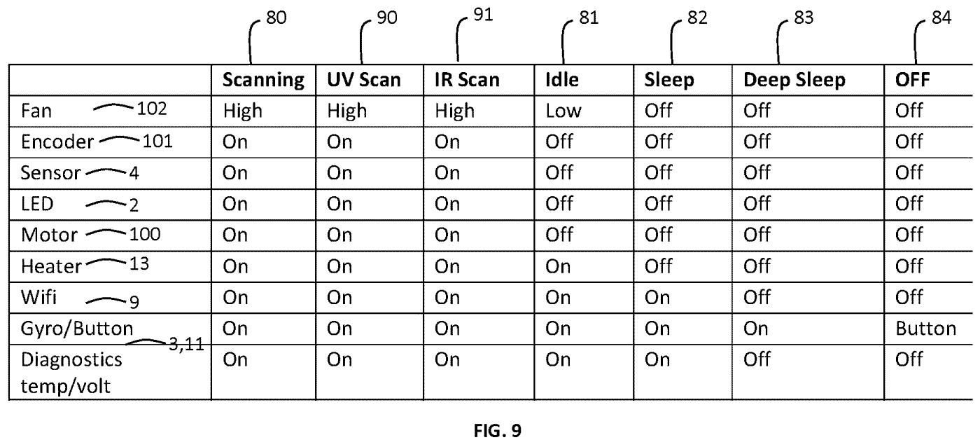

FIGS. 7A, 7B and 7C, illustrate an example where the power management unit 5 is configured to set the intraoral scanner 1 in the scanning mode if the motion corresponds to a predefined direction or pattern that relates to an intentionally use of the intraoral scanner 1. The direction or the pattern may be determined in relation to a first longitudinal axis 71 that propagates in a longitudinal direction of the scanner 1. Furthermore, a reference axis 70 is needed. The reference axis 70 propagates in a longitudinal direction of the scanner 1 when the scanner 1 is placed on table or a cradle. The reference axis 70 may be calibrated when no motion is detected or during booting of the scanner 1. The direction and/or movement pattern of the scanner 1 may be determined by an angle 72 between the first longitudinal axis 71 and the reference axis 70. In FIG. 7A, the angle 72 is about zero indicating that the scanner is in a resting position, i.e. not being used intentionally. In FIG. 7B, the scanner 1 is tilted such that angle 72 is below a minimum angle (θmin), such as −5 degrees and wherein the minimum angle is −4 degrees, and in FIG. 7C, the scanner 1 is tilted such that the angle 72 is above a maximum angle (θmax). In the examples illustrated in FIGS. 7B and 7C, the tilting angle span being wider than the angle span defined by the minimum angle (θmin) and the maximum angle (θmax) would results in an activation of the scanner 1, i.e. the scanner entering the scanning mode. While walking with the scanner the angle span of the tilting is in most cases within the angle span defined by the minimum and maximum angle, and thereby, the scanner 1 would not be activated, i.e. the scanner stays in a low power mode. In another example, the scanner 1 may be tilted such that the angle 72 is about 90 degrees resulting in an activation of the scanner 1. In both examples the direction and/or the movement pattern of the scanner 1 sets the scanner 1 into a scanning mode. FIG. 8 illustrates a state diagram of the different power modes which can be set by the power management unit 5. The scanner 1 may be in the scanning mode 80 which either is in a normal scanning mode where the projector unit is configured to emit white light, or in a fluorescence mode where the projector unit is configured to emit colored light, or in a none-visible mode where the projector unit is configured to emit infrared or near-infrared light, or in a mode where the projector unit is configured to emit light of white, colored and/or IR/NIR wavelengths. Via a button press or an external device 80A, such as a computer that includes an application for communicating with the scanner 1, the power management unit 5 sets the scanner 1 into the idle mode 81, or back from the idle mode 81 to the scanning mode 80. The motion sensor 3 detects no movements or movements below a motion threshold 23 during a period of seconds, and the power management unit 5 is then configured to set the scanner 1 into a sleep mode 82 after being in the idle mode 81. Again, via the user interface 11, such as button press or the external device, the power management unit 5 is configured to set the scanner 1 back to the idle mode 81 from the sleep mode 82. Furthermore, the motion sensor 3 detects not movement or movements below a motion threshold 23 during a period of seconds, and the power management unit 5 is then configured to set the scanner 1 into a deep sleep mode 83, and back to the sleep mode 82, if the accelerometer 3 detects a movement of the scanner, or, if the user interacts with the user interface 11, such as pressing a button on the scanner 1. If the scanner 1 is not moved during further seconds then the scanner 1 is set into an off mode 84 by the power management unit 5. FIG. 9 describes the settings of the different components (2, 3, 4, 9, 11, 13, 100, 101, 102) of the scanner 1 in the different modes. The encoder is a video encoder.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is Intended that the structural features of the devices described above, either in the detailed description and/or in the claims, may be combined with stops of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. An intraoral scanner comprising:
a projector unit configured to emit light at least onto a dental object of a patient;
an image sensor configured to acquire reflected light from at least the dental object;
a battery for powering the intraoral scanner;
a processor unit configured to process the reflected light into one or more 2D images and/or 3D images;
a wireless interface configured to communicate with an external device the one or more 2D images and/or 3D images,
a motion sensor configured to sense a motion of the intraoral scanner;
a timer unit;
a temperature sensor configured to produce a temperature signal that includes a temperature of the intraroral scanner, and
a power management unit configured to reduce the power consumption of the intraoral scanner based on a motion signal provided by the motion sensor, a timer signal provided by the timer unit, and the temperature signal, a and wherein the motion signal includes information about motion of the intraoral scanner, wherein the motion is below a motion threshold during one or more time periods, and after each of the one or more time periods the power consumption is reduced gradually, and, the power consumption is reduced when the temperature of the intraoral scanner is above a temperature threshold.

2. The intraoral scanner according to claim 1, wherein the power consumption reduction includes setting the intraoral scanner into different power modes based on the motion signal and the timer signal after each of the one or more time periods.

3. The intraoral scanner according to claim 1, wherein the power consumption reduction includes gradually turning off components of the intraoral scanner after each of the one or more time periods.

4. The intraoral scanner according to claim 3, wherein the power consumption reduction includes gradually turning off one or more groups of components of the intraoral scanner after each of the one or more time periods, and wherein each of the one or more groups includes one or more components of the intraoral scanner.

5. The intraoral scanner according to claim 1, wherein the motion sensor includes at least an accelerometer and a gyroscope.

6. The intraoral scanner according to claim 2, wherein the different modes include:
a scanning mode, wherein the motion signal includes information about motion of the intraoral scanner being above the motion threshold,
an idle mode, wherein the motion signal includes information about motion of the intraoral scanner that is below the motion threshold during a first time-period,
a sleep mode, wherein the motion signal includes information about motion of the intraoral scanner that is below the motion threshold during a second time period, wherein the second time period is longer than the first time period, and.
a deep sleep mode, wherein the motion signal includes information about motion of the intraoral scanner that is below the motion threshold during a third time period,
wherein the third time period is longer than the second time period, and wherein the first time-period, the second time period and the third time period are part of the one or more time periods.

7. The intraoral scanner according to claim 6, wherein the intraoral scanner includes components and/or one or more groups of components, and the components include at least the projector unit, the image sensor, processor unit, the wireless interface, the motion sensor, the timer unit, and the power management unit.

8. The intraoral scanner according to claim 6, wherein the scanning mode includes that all components are turned on.

9. The intraoral scanner according to claim 6, wherein the idle mode includes that at least the projector unit, the image sensor and the processor unit are turned off.

10. The intraoral scanner according to claim 9, wherein the idle mode includes that a heater of the intraoral scanner is configured to heat at a lower temperature than in the scanning mode.

11. The intraoral scanner according to claim 6, wherein the sleep mode includes that at least the image sensor, the projector unit, the processor unit and the fall heater are turned off.

12. The intraoral scanner according to claim 6, wherein the deep sleep mode includes that at least the projector unit, the image sensor, the processor unit, the wireless interface, and part of the motion sensor are turned off.

13. The intraoral scanner according to claim 5, wherein the gyroscope is turned off and the accelerometer is on.

14. The intraoral scanner according to claim 6, comprising a user interface, and when the power management unit receives a user interface signal from the user interface, the power management unit is configured to set the intraoral scanner into the scanning mode if the intraoral scanner is in the sleep mode or in the deep sleep mode.

15. The intraoral scanner according to claim 6, wherein the motion is above the motion threshold, the power management unit is configured to set the intraoral scanner into the scanning mode if the intraoral scanner is in the idle mode.

16-32. (canceled)
